(19) United States
(12) Patent Application Publication
(10) Pub. No.: US 2001/0020470 A1
(43) Pub. Date: Sep. 13, 2001

(54) Portable,multipurpose, air dispensing apparatus

(76) Inventor: Andy Zupan, Tajares, NM (US)

Correspondence Address: Law Offices of John D. Gugliotta, PE., Esq., 202 Delaware Building, 137 South Main Street, Akron, OH, 44308, US

(21) Appl. No.: 09/778,544

(22) Filed: Feb. 7, 2001

Related U.S. Application Data
(63) Continuation of application No. 09/264,699, filed on Mar. 8, 1999, now abandoned.

(51) Int. Cl. A62B 7/00; A62B 9/00; A61M 15/00; A61M 16/00; A62B 18/00

(52) U.S. Cl. 128/200.24

(57) ABSTRACT

A portable, multipurpose, air dispensing apparatus is disclosed, comprised of a small, portable canister that holds a combination of compressed oxygen and nitrogen that the user can tap in order to recuperate from the physical exertion by delivering these gasses to the cardiovascular system in a quick and efficient manner. The canister includes a mouthpiece/regulator combination that the user places in his or her mouth and, by depressing a button, a breath of the pure oxygen/nitrogen is delivered. The canister is disposable and replaceable and holds enough gas for approximately 15 average breaths. Carried in a convenient arm band, the canister gives the user the ability to tap the benefits of the gasses whenever the need is felt. Additionally, the present invention serves a safety purpose in high-altitude sports where the air is thin and oxygen supplies are low.

### RELATED APPLICATIONS AND DISCLOSURES

The present invention is a Continuation in Part of U.S. application Ser. No. 09/264,699, filed on Mar. 8, 1999.

### BACKGROUND OF THE INVENTION

1. Field of the Invention

The present invention relates generally to air tanks, and, more particularly, to a portable, multipurpose, air dispensing apparatus.

2. Description of the Related Art

High intensity sports, exercises and other recreational activities are extremely popular these days. Whether one is involved in a traditional sports such as football or basketball, in high intensity exercises such as aerobics or running, or in a more modern or extreme sports like in-line skating, mountain biking and water sports, a high degree of physical exertion takes place.

As a result of these activities, the body can be depleted of oxygen as the cardiovascular system is pressed to keep up with the level of physical exertion. Accordingly, there is a constant need for exercise or health aids that help the user to reduce the physical impact that their activities have on their bodies and allow them to recover quickly so that they can participate in yet more activities.

Any type of portable oxygen or air dispenser must be portable, and therefore, not interfere with the activities of the potential user. Also, the device must be stored in a place where it can be easily found and used, since the device may be needed during an emergency situation, such as during drowning, where the person may be partially mentally impaired.

In the related art, several devices disclose portable, self-contained oxygen dispensers or breathing apparatuses. These include U.S. Pat. No. 5,620,664, issued in the name of Palmer, U.S. Pat. No. 4,409,978, issued in the name of Bartos, U.S. Pat. No. 4,154,236, issued in the name of Eckstein et. al., U.S. Pat. No. D 372,529, issued in the name of Remes and U.S. Pat. No. D297,869, issued in the name of Bartos.

Several patents describe an emergency escape breathing apparatus. These include U.S. Pat. No. 5,318,019, issued in the name of Celaya and U.S. Pat. No. 4,221,216, issued in the name of Kranz.

U.S. Pat. No. 4,669,461, issued in the name of Battaglia et. al., discloses a device for administering oxygen to infants while nursing.

U.S. Pat. No. 4,423,723, issued in the name of Winkler et. al., discloses a closed-cycle respirator with an emergency oxygen supply.

U.S. Pat. No. D 311,679, issued in the name of Morris, discloses the ornamental design of a holder for an oxygenator bottle.

The problem with many of these devices is that they are bulky, and are therefore, limited in use. Also, the devices are not specifically designed for use during non sporting activities, and are not portable enough to be carried in a purse or pocket. As such, executives needing oxygen before a speech, etc. are not contemplated by the prior art devices. The devices in the prior art have numerous components, and as such, are subject to component failure. These devices are also heavy, which reduces portability.

Many of the devices are designed to function only in oxygen deprived atmospheres, and as such must be bulky and heavy. They do not contemplate supplemental or complementary air supplies during non-emergency situations, such as when only one or two breaths are needed.

Also, not all of these devices are designed to release just one breath of air at a time.

A search of the prior art did not disclose any patents that anticipate directly many features of the instant invention.

Consequently, a need has been felt for providing an apparatus and method which overcomes the problems cited above.

### SUMMARY OF THE INVENTION

It is therefore an object of the present invention to provide an improved portable, multipurpose, air dispensing apparatus that is easily stored and activated to provide a short term supplemental air supply.

Briefly described according to one embodiment of the present invention, a portable, multipurpose, air dispensing apparatus is disclosed, comprising a small, portable canister that holds a combination of compressed oxygen and nitrogen that the user can tap in order to recuperate from the physical exertion by delivering these gasses to the cardiovascular system in a quick and efficient manner. The canister includes a mouthpiece/regulator combination that the user places in his or her mouth and, by depressing a button, a breath of the pure oxygen/nitrogen is delivered. The canister is disposable and replaceable and holds enough gas for approximately 15-90 average breaths, depending on exact canister size and PSI rating. Carried in a convenient arm wrap, the canister gives the user the ability to tap the benefits of the gasses whenever the need is felt. Additionally, the present invention serves a safety purpose in high-altitude sports where the air is thin and oxygen supplies are low.

It is another object of the present invention to provide a device that can provide supplemental oxygen during events such as rafting, swimming, surfing, boating, skiing, sky diving, hang gliding, life guarding, in line skating, mountain biking, jogging, business functions, such as speeches.

It is another object of the present invention to provide a device that can be stored in a pocket, and therefore can be used in public, when required.

It is another object of the present invention to provide a device that adjustably fits around a user's arm or leg, thus ensuring quick access to the device during emergency situations.

DESCRIPTIVE KEY

| | | | |
|---|---|---|---|
| 10 | portable, multipurpose, | 66 | outer chamber |
| | air dispensing | 67 | conduit |
| | apparatus | 68 | valve housing |
| 20 | canister | 69 | flexible tube |
| 30 | regulator | 70 | female threads |
| 40 | mouthpiece | 71 | piston |
| 45 | mouth | 72 | lever arm |
| 50 | activation button | 73 | pivot mound |
| 51 | threaded shank | 75 | male threads |
| 52 | constant volume discharge | 80 | sealing means |
| | valve | 83 | puncture stem |
| 53 | seat member | 90 | canister securing means |
| 54 | annular seat | 100 | arm |
| 55 | thumb | 110 | arm wrap |
| 56 | ball | 120 | arm wrap securement means |
| 57 | hand | 125 | interior surface |
| 58 | metal seat | 130 | nonslip surface |
| 59 | spring | 140 | canister pouch |
| 60 | female inlet | 150 | flap |
| 61 | metal pin | 153 | opening |
| 62 | valve arm | 160 | flap securement means |
| 63 | coil spring | 165 | flap |
| 64 | chamber | | |
| 65 | orifice passageway | | |

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a perspective view of the preferred embodiment of portable, multipurpose, air dispensing apparatus 10;

FIG. 2 is a side view thereof;

FIG. 3 is a top plan view thereof;

FIG. 4 is an in-use view thereof;

FIG. 5 is an exploded view thereof;

FIG. 6 is a bottom view thereof;

FIG. 7 is a perspective view of the canister securement means;

FIG. 8 is a perspective view of the canister securement means shown in-use with a human arm;

FIG. 9 is an in-use view of another embodiment of the canister securement means;

FIG. 10 is a cross-sectional view as taken along lines X-X of FIG. 2; and,

FIG. 11 is a sectional view of the constant volume discharge valve according to the preferred embodiment of the present invention.

### DESCRIPTION OF THE PREFERRED EMBODIMENTS

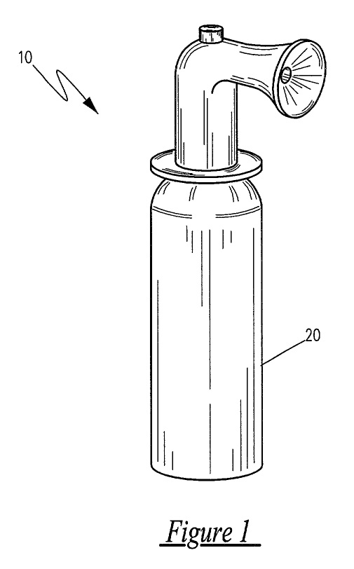

The best mode for carrying out the invention is presented in terms of its preferred embodiment, herein depicted within the FIGS. 1 through 8.

1. Detailed Description of the Figures

Referring now to FIG. 1, a portable, multipurpose, air dispensing apparatus 10 is shown, according to the present invention, comprises a small, portable canister 20 that holds a combination of compressed oxygen and nitrogen.

The canister 20 is of a linearly elongated, cylindrical, hollow configuration, and is constructed of a lightweight metal, such as aluminum or steel.

The present invention is designed to be used to facilitate speedy recuperation from the physical exertion by delivering these gasses to the cardiovascular system in a quick and efficient manner. It is envisioned that the canister holds enough gas for approximately 15 average breaths.

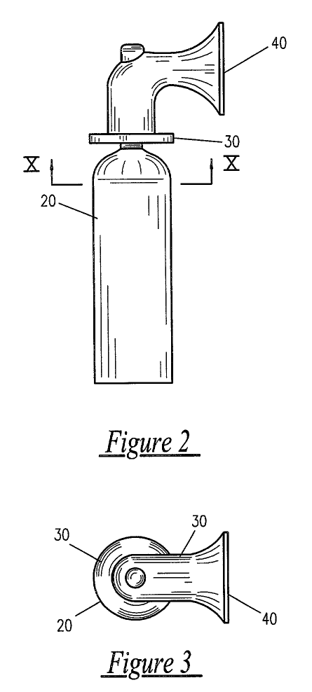

Referring now to FIG. 2, located on the top of the canister is a regulator 30, and above the regulator 30, a mouthpiece 40, over which the user places in his or her mouth 45 (not shown in FIG. 2).

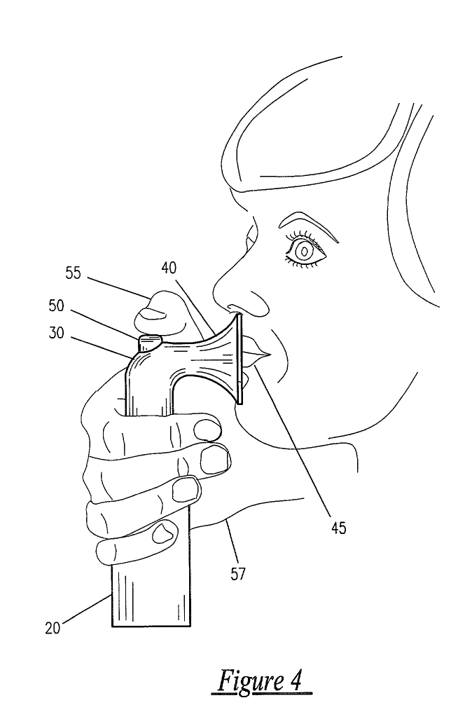

Referring now to FIGS. 3 and 4, an activation button 50 is located on the top of the regulator 30. By depressing the activation button 50, the regulator 30 releases the equivalent of one breath of the oxygen nitrogen mixture through the mouthpiece 40 and into the mouth 45 (not shown in FIG. 3) of the user.

A constant volume discharge valve 52 (shown in FIG. 11) is located inside a valve housing 68 of the regulator 30. The valve housing 68 is formed of metal having the constant volume discharge valve 52 machined therein. A threaded shank 69 for capturing a piston 71 and its essential components is threadably received in the valve housing 68. The discharge valve 52 includes a biased valve closure member defined by the piston 71 having a supple and sealing seat member 53 engaging an annular seat 54.

The discharge valve 52 further includes a ball 56 for engaging a hard metal seat 58 for acting as a pressure control means. The ball 56 is biased in a closed position by internal pressure of the canister 20 and a resilient spring 59. The hard metal seat 58 prevents a sealing seat from being achieved. As the supply pressure of the oxygen nitrogen mixture decreases, the ball 56 is lifted from the hard metal seat 58 by a metal pin 61 being resiliently biased through a valve arm 62 and a resilient coil spring 63. The valve arm 62 thereby functions as a pressure regulator for maintaining a substantially constant pressure within a chamber 64.

The valve arm 62 has a fixed diameter orifice passageway 65 communicating from the chamber 64 to an outer chamber 66 thereby providing open communication through a conduit 67 and a flexible tube 69 to the puncture stem 83 (not shown). The valve arm 62 is actuated by a lever arm 72 resting above a pivot mound 73 and being coupled at one end to the valve arm 62 and at an opposite end to the lower surface of the activation button 50. Pressure exerted on the activation button 50 actuates the lever arm 72 to pivot about the mound 73 thereby lifting the lever arm 72 for releasing a preset amount of the oxygen nitrogen mixture.

The valve arm 62 thus further functions to release a preset amount of the oxygen nitrogen mixture equivalent to one human breath, each time the activation button 50 is depressed.

The present invention is configured and designed for activation via a human thumb 55 (not shown in FIG. 3). The size and configuration of the present invention allows holding and activation of the canister 20 with only one hand 57.

It is envisioned that the canister 20 is sufficiently small so as to be portable, capable of easy grasping, holding and activation with one hand 57, and capable of being carried in a pocket. As such, the present invention can be used anywhere it is needed. For purposes of disclosure, the canister 20 is depicted as being 1 inch in radial diameter, and 3 and ½ inches in height. This configuration allows for optimal usage, while other sizes are also envisioned. The canister 20 size can range from 5 and ½ inches to 15 inches, depending on the needs of the user.

It is envisioned that the simple configuration ensures fewer component failures. The small, lightweight design ensures ease of use anywhere during any activity.

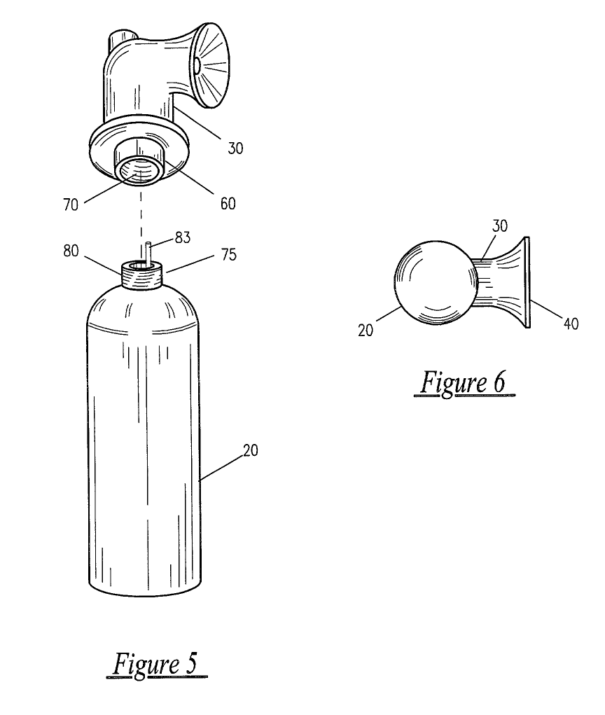

Referring now to FIG. 5, the regulator 30 is attached to the top of the canister 20 via a female inlet 60 and female threads 70, located on the bottom of the regulator 30. These female threads mate with male threads 75 located on the top of the canister. Sealing means 80, such as an O-ring, ensure an air tight seal between the regulator 30 and the canister 20. A conventional puncture stem 83 on the top of the canister 20 allows air to be dispensed from the canister 20 once attached to the regulator 30.

As such, the canister 20 is releasably secured to the regulator 30 and thereby the mouthpiece 40. Being disposable, the canister 20 can be replaced when depleted.

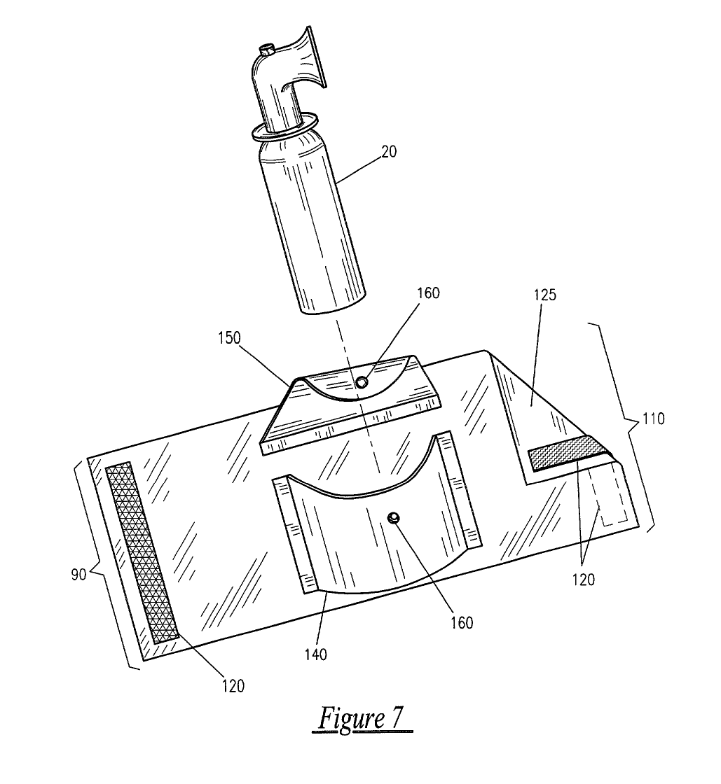

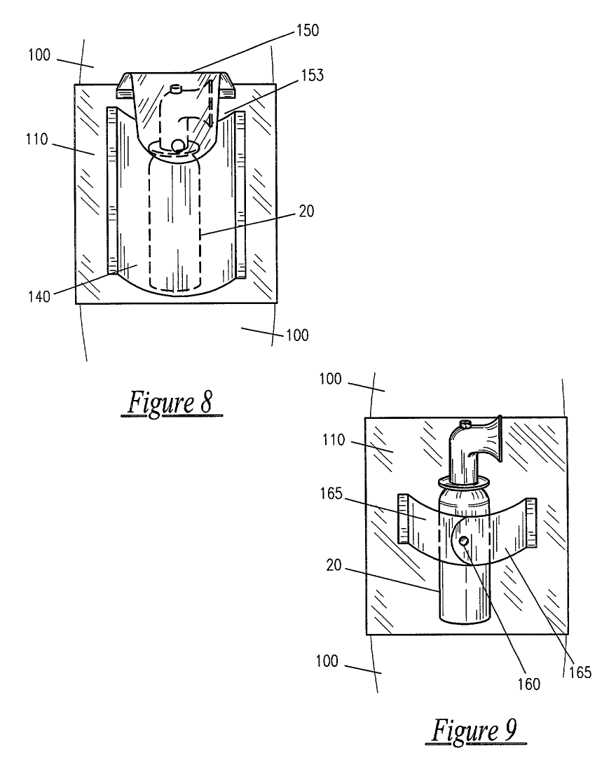

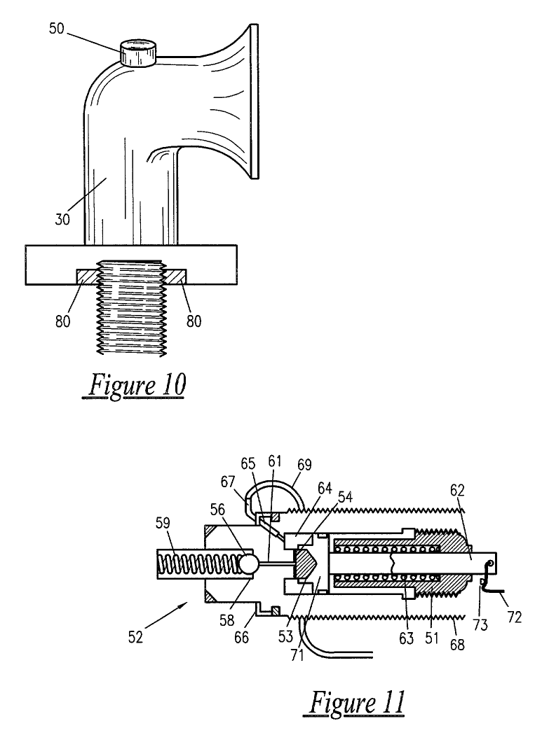

Referring now to FIGS. 7 and 8, the present invention is releasably secured to the body of the user via a convenient canister securement means 90. The canister securement means 90 is designed to releasably secure the canister 20 to the arm 100 or leg of the user, within easy grasp of the user. The canister securement means 90 eliminates the need to search for the canister 20, especially during times where time is of the essence, or mental functioning is impaired, such as during emergencies involving water.

For purposes of disclosure, the canister securement means 90 is depicted as an adjustable arm wrap 110, of a generally rectangular configuration. The arm wrap 110 is constructed of a strong, lightweight, elastic material, such as neoprene or cloth. The arm wrap 110 is wrapped around the arm 100 of the user, and adjustably secured via arm wrap securement means 120, such as a hook and loop fastener. The interior surface 125 of the arm wrap 110, which rests against the arm 100 of the user, contains a non-slip surface to reduce movement of the arm wrap 110 along the arm 100 during activity.

A canister pouch 140 is located on the exterior surface of the arm wrap 110. The canister pouch 140 is configured and sized so as to snugly hold the canister 20 within itself. When the canister 20 is in the canister pouch 140 and the canister pouch 140 secured to the user's arm 100, the elongated centerline of the canister 20 is parallel to the elongated centerline of the arm 100, so as to facilitate easy grasping by the opposite arm 100 when needed.

It is also envisioned that a flap 150 is attached to the exterior surface of the arm wrap 110, and covers the opening 153 in the canister pouch 140. The flap 150 is releasably secured against the exterior surface of the canister pouch 140 via flap securement means 160, such as a hook and loop fastener or a snap fastener. The flap securement means 160 is designed to be easily and quickly disconnected to allow easy access to the canister 20.

The canister pouch 140 does not completely cover the canister 20 once inserted, so that when the flap 150 is released, the canister 20 can be easily grasped by the top. For intense activities, the canister pouch 140 may be altered so that the canister 20 fits completely inside the canister pouch 140, like a knife in a sheath.

Referring now to FIG. 9, in an alternate embodiment of the present invention, the canister 20 is releasably secured to the exterior surface of the arm wrap 110 via a flap 165 and flap securement means 160. The canister pouch 140 is not included.

The present invention is designed to be used during all water activities as well as when the air is thin, such as during sky diving or mountain climbing. The portable nature also facilitates use during everyday activities, such as when a person is nervous giving a speech, where the canister 20 can be stored in a purse or pocket.

The present invention is also designed to serve as an emergency air supply for those who use oxygen on a long term basis, such as asthmatics. The present invention may be used as a temporary air supply, and as such, would be useful in first aid kits, extinguisher compartments, and the like.

As such, it is envisioned that the present invention can be used to provide supplemental oxygen during events such as rafting, swimming, surfing, boating, skiing, sky diving, hang gliding, life guarding, in line skating, mountain biking, jogging, and during business functions, such as speeches.

In an alternate embodiment of the present invention, the mouthpiece 40 and regulator 30 are permanently affixed to the pre-filled canister 20, and as such, the entire device is disposable when the canister 20 is depleted. This reduces the need to store the device after use.

It is envisioned that other styles and configurations of the present invention can be easily incorporated into the teachings of the present invention, and only one particular configuration shall be shown and described for purposes of clarity and disclosure and not by way of limitation of scope.

2. Operation of the Preferred Embodiment

Referring now to FIG. 6, to use the present invention, the canister 20 is secured to the person's arm 100 or leg via the canister securement means 90. When air is needed, the user releases the flap securement means 160, grabs the canister 20 with any free hand 57 and places the mouthpiece 40 against his or her mouth 45. Next, the user places his thumb 55, from the hand 57 holding the canister 20, onto the top of the canister 20 and depresses the activation button 50. A supply of air equivalent to one breath of air enters the user's mouth 45. Each time a breath is needed, the activation button 50 is depressed. In the preferred embodiment, when the canister 20 is depleted of oxygen, the canister 20 can be unscrewed from the regulator 30, and replaced. In the alternate embodiment, the entire device is discarded.

The foregoing description is included to illustrate the operation of the preferred embodiment and is not meant to limit the scope of the invention. The scope of the invention is to be limited only by the following claims.

What is claimed is:

1. A portable, multipurpose, air dispensing apparatus comprising:
a small, portable canister of a linearly elongated, cylindrical, hollow configuration, said canister holding a combination of compressed oxygen and nitrogen;
a regulator, said regulator located on the top of said canister, and said regulator having a valve housing disposed within said regulator;
a mouthpiece, said mouthpiece located above said regulator, and designed for the user to place his or her mouth over said mouthpiece;
an activation button, said activation button located on the top of said regulator; said activation button designed to be depressed, and with each depression, to release the equivalent of one breath of the pure oxygen/nitrogen through said mouthpiece and into said mouth of the user; and
canister securing means, said canister securing means designed to releasably secure said canister to an appendage of a user.
a constant volume discharge valve, said constant volume discharge valve located inside said valve housing of said regulator.

2. The portable, multipurpose, air dispensing apparatus described in claim 1, wherein said canister holds enough gas for approximately 15-90 average breaths, depending on canister size and PSI rating.

3. The portable, multipurpose, air dispensing apparatus described in claim 1, wherein said regulator further comprises a constant volume discharge valve, said constant volume discharge valve releasing a preset amount of air equivalent to one human breathe, each time said activation button is depressed.

4. The portable, multipurpose, air dispensing apparatus described in claim 1, wherein said present invention is configured and designed to be held and activated by one hand, with the thumb depressing said activation button.

5. The portable, multipurpose, air dispensing apparatus described in claim 1, wherein said regulator further comprises:
a female inlet and female threads, located on the bottom of said regulator, said female threads designed to mate with male threads located on said top of said canister, thereby making said canister detachable from said regulator; and
sealing means, said sealing means designed to ensure an air tight seal between said regulator and said canister.

6. The portable, multipurpose, air dispensing apparatus of claim 5, wherein said canister is designed to be replaced when depleted.

7. The portable, multipurpose, air dispensing apparatus described in claim 1, wherein said canister, mouthpiece and regulator are sufficiently small so as to be portable, capable of easy grasping, holding and activation with one hand, and capable of being carried in a pocket.

8. The portable, multipurpose, air dispensing apparatus of claim 7, wherein said canister is 1 inch in radial diameter, and 3 and ½ inches in height.

9. The portable, multipurpose, air dispensing apparatus described in claim 1, wherein said canister securing means further comprises;
an adjustable arm wrap, of a generally rectangular configuration, said arm wrap being constructed of a strong, lightweight, elastic material, and designed to be wrapped around an appendage of the user;
a non-slip surface, said non-slip surface located on the interior surface of said arm wrap, said arm wrap adapted to rest against the user's skin;
a canister pouch, said canister pouch located on the exterior surface of said arm wrap, and configured and sized so as to snugly hold said canister within itself through an opening in said canister pouch; and
flap securement means, said flap securement means releasably secures said flap to said exterior surface of said canister pouch.

10. The portable, multipurpose, air dispensing apparatus of claim 9, wherein said flap securement means is a hook and loop fastener.

11. The portable, multipurpose, air dispensing apparatus of claim 9, wherein said flap securement means is a snap fastener.

12. The portable, multipurpose, air dispensing apparatus of claim 9, wherein when said canister is in said canister pouch and said canister pouch secured to the user's arm, the elongated centerline of said canister is parallel to the elongated centerline of said arm, so as to facilitate easy grasping by the opposite arm when needed.

13. The portable, multipurpose, air dispensing apparatus of claim 9, wherein said canister pouch does not completely cover said canister once inserted, so that when said flap is released, said canister can be easily grasped by a top of said canister.

14. The portable, multipurpose, air dispensing apparatus of claim 9, wherein said arm wrap is constructed of neoprene.

15. The portable, multipurpose, air dispensing apparatus of claim 9, wherein said canister is releasably secured to said exterior surface of said arm wrap via said flap and flap securement means.

16. The portable, multipurpose, air dispensing apparatus described in claim 1, wherein said mouthpiece and regulator are permanently affixed to said canister.

17. The portable, multipurpose, air dispensing apparatus described in claim 1, wherein said constant volume discharge valve comprises:
a threaded shank, said threaded shank for capturing a piston, and said threaded shank being threadably received in said valve housing; wherein said piston having a supple and sealing seat member engaging an annular seat; and
a ball, said ball for engaging a hard metal seat for providing a pressure control means, said ball being biased in a closed position by internal pressure of said canister and a resilient spring, and said hard metal seat for preventing a sealing seat from being achieved.

18. The portable, multipurpose, air dispensing apparatus described in claim 17, wherein said ball is lifted from said hard metal seat by a metal pin being resiliently biased through a valve arm and a resilient coil spring as supply pressure of the oxygen nitrogen mixture decreases, wherein said valve arm functions as a pressure regulator for maintaining a substantially constant pressure within a chamber.

19. The portable, multipurpose, air dispensing apparatus described in claim 18, wherein said valve arm has a fixed diameter orifice passageway communicating from said chamber to an outer chamber for providing open communication through a conduit and a flexible tube to a puncture stem, said valve arm being actuated by a lever arm resting above a pivot mound.

20. The portable, multipurpose, air dispensing apparatus described in claim 19, wherein said lever arm being coupled at one end of said valve arm and at an opposite end to a lower surface of said activation button, said lever arm pivoting about said mound to lift said lever arm for releasing a preset amount of oxygen nitrogen mixture after pressure is exerted on said activation button.
